(19) United States
(12) Patent Application Publication
(10) Pub. No.: US 2011/0152713 A1
(43) Pub. Date: Jun. 23, 2011

(54) System for Recording Measured Values in or on an Organism, and Method for Producing a Componet of this System

(76) Inventors: Heribert Stephan, Muenchen (DE); Werner Kraus, Muenchen (DE); Stephanie Kraus, Muenchen (DE)

(21) Appl. No.: 13/054,694

(22) PCT Filed: Jul. 17, 2009
(86) PCT No.: PCT/DE09/01000

§ 371 Date: Mar. 8, 2011

(30) Foreign Application Priority Data
Jul. 18, 2008 (DE) 10-2008-033-805.2

(51) Int. Cl. A61B 5/053

(52) U.S. Cl. 600/547

(57) ABSTRACT

A system for detecting measured values in or on an organism (10) comprising at least one secondary device (12) attachable to the organism or implantable in the organism, the impedance of which depends on a state of the surroundings of the implantable secondary device, a coil arrangement (14, 16) placeable outside the organism that acts as a primary device for generating an electromagnetic alternating field in the vicinity of the secondary device in an implanted state, and an evaluation device (20) placeable outside of the organism for detecting and analysing measured values depending on the impedance of the secondary device, wherein the secondary device comprises electrically conductive means (22) which are applied using thin film technology.

The invention further relates to a method for producing a system component.

The invention relates to a system for detecting measured values in or on an organism.

The invention further relates to a method for producing a component of such a system.

Cardiovascular disease is the leading cause of death in the industrialised world. Globally, more than 1.5 million interventions for widening constricted or occluded blood vessels are carried out each year. One example of these interventions is percutaneous transluminal coronar angioplasty (PTCA). In some of these interventions, stent grafts, commonly referred to as stents, are implanted. The success of these measures, however, is often affected by the high probability of restenosis. Approximately 30 to 50% of patients who undergo balloon dilatation and approximately 22 to 30% of patients with stents suffer restenosis, i.e., a renewed constriction of the blood vessels within six months of the intervention. In 2000 in Germany alone, 25,000 patients had to be reoperated upon, generating costs of approximately 500 million Euros.

In order to identify restenosis at an early stage, i.e. particularly before a detrimental or total occlusion of the blood vessel occurs, it is useful to diagnose the state of the blood vessel in the affected area after an operation. However, the current diagnosis methods for detecting vascular changes are based on the detection of a reduced blood flow or coarse changes to the arterial walls using imaging methods. These diagnosis methods can, due to their principle, only show advanced stages of the changes. The invasive administration of contrast agents which often accompanies these imaging methods may lead to complications which may result in pain, perforation of the arteries, arrhythmias, and, in the worst case, heart attacks and strokes.

Infections are another problem in connection with the implantation of a stent graft or a vascular implant. A high percentage of these infections will lead to serious and life-threatening situations. Often, even administering high dosages of antibiotics for a prolonged period of time does not result in successful therapy. Finally, there remains no alternative to exchanging the vascular implant, which renews the associated operation risks.

The above named problem with infections does not only exist in connection with stent grafts or vascular implants but also in connection with other implants, for example, osteosynthesis means or endoprostheses of joints or other skeletal parts. Here, infections are frequently caused by the development of biofilms on the implants which, for example, often host the difficult to treat multi-resistant staphylococcus aureus (MRSA).

Therefore, the object of the present invention is the early recognition of an imminent or incipient change in the area of implants by means of non-invasive measures and the provision of the technical devices required for this purpose.

Said object is solved by the features of the independent claims.

Advantageous embodiments of the invention are specified in the dependent claims.

The invention consists of a system for detecting measured values in or on an organism comprising at least one secondary device attachable to the organism or implantable into the organism, the impedance of which depends on a state of the surroundings of the implantable secondary device, a coil arrangement placeable outside the organism that acts as the primary device for generating a electromagnetic alternating field in the area of the secondary device in an implanted state, and an evaluation device placeable outside the organism for detecting and analysing measured values depending on the impedance of the secondary device, wherein the secondary device comprises electrically conductive means applied using thin film technology. Owing to thin film technology, virtually any implant can be provided with electric properties so that it can interact with an electromagnetic alternating field generated by a coil arrangement outside the organism. This interaction can be detected outside of the organism in various ways in order for changes in the region of the secondary device, in particular, an implant to be detected in a non-invasive manner. For example, both hyperplasias, i.e., an excessive cell growth inside stents and the incipient formation of biofilm on an implant can be detected in this manner.

Usefully, it is contemplated that the secondary device comprises an electric oscillating circuit, the impedance and resonant frequency of which depend on the state of the surroundings of the secondary device. If the externally generated electromagnetic alternating field meets the resonant frequency, it can be detected and analysed by the evaluation device. If the surroundings of the secondary device change due to cell growth, a change of the cross section in the blood vessel, or biofilm formation, the impedance of the oscillating circuit will be affected, and its resonant frequency will change. Therefore, the evaluation device outside the organism can detect a change in the area of the implant and thus indicate an imminent complication.

The system according to the invention is, in a particularly useful manner, further developed in that the electrically conductive means comprise first electrically conductive means applied to the implantable secondary device which constitute a component of an oscillating circuit, an electrically insulating layer is applied to the first electrically conductive means, a second electrically conductive means which constitute another component of the oscillating circuit are applied to the electrically insulating layer, and the first electrically conductive means are contacted by the second electrically conductive means so that the oscillating circuit is formed. Thus, the required components of an oscillating circuit can be precisely applied to the implant using thin film technology. The inductivity and capacity of the oscillating circuit can be varied depending on the design of the electrically conductive means.

It is particularly useful that the first electrically conductive means exhibit an exterior with windings and an interior with capacitive properties.

According to another embodiment of the invention, it may be contemplated that the secondary device comprises a measuring device detecting measured values depending on the state of the tissue surrounding the secondary device, the secondary device comprises a transmitter device transmitting signals depending on the measured values, and a receiver device placeable outside the organism is provided which receives signals transmitted by the transmitter device and supplies them to the evaluation device. While the embodiments of the invention described so far are based on the variation of the resonant frequency of an oscillating circuit, it may also be contemplated to provide the secondary device with a measuring device which detects the various characteristics in the device's surroundings by means of sensors. For example, the pH-value in the vicinity of the implanted device can provide information on changes, for instance in the case of biofilm formation. One example of such a sensor is the ion-sensitive MOS-FET (ISFET). In this transistor, the common polysilicon gate is replaced by a sensor-specific metallization. The use of different materials renders possible the realisation of sensors which are sensitive to gasses or ion concentrations in liquid solutions. These components serve as basic structures for biosensors. Signals corresponding to the values detected by the measuring device may then be transmitted by a transmitter device and analysed outside of the organism. In the present case, the use of the alternating field outside of the organism primarily serves to transport the power required for the operation of the measuring device into the organism.

Usefully, it is contemplated that transmitter device comprises at least one RFID transponder. A RFID transponder is a device which can only “transmit” information in cooperation with an evaluation device or, respectively, a reading device which is realised by the receiver device. Ultimately, the RFID transponder will receive an electromagnetic high frequency field generated by the evaluation device or the reading device in order to then change said field depending on information stored in the RFID transponder. This change is detected by the evaluation or reading device. As the function of an RFID transponder is limited in comparison to conventional active transmitters, it is both cost-effective and space-saving.

The information transfer from the RFID transponder to the evaluation device may take place according to the principle that the readable information content of the RFID transponder is changeable depending on measured values provided by the measuring device. In the simplest case, different voltages are applied to the memory of the RFID transponder by the measuring device, and said voltages reflect the characteristics detected by the measuring device. Different voltages can ensure that the content of the memory of the RFID transponder is changed so that, ultimately, the identification transmitted to the evaluation device by the RFID transponder is also changed. In order to ensure the option to change the content of the memory of the RFID transponder, it is necessary to use writable RFID transponders.

Alternatively or additionally, it is also possible that a plurality of RFID transponders is provided which are activatable or deactivatable depending on the measured values provided by the measuring device. In this case, non-writable transponders are sufficient. One or more threshold value circuits in which the measuring device and the RFID transponder are integrated ensure that different RFID transponders are active or inactive depending on the voltage supplied by the measuring device. In this way, the evaluation device can receive different identifiers depending on the voltage applied by the measuring device and, on this basis, ensure that the corresponding information is transmitted to the evaluation device located outside the organism.

The invention further consists of a method for producing a secondary device in which an electric voltage is inducible by means of a primary device, comprising the steps of providing an implant, applying first electrically conductive means on the implant which are to constitute a component of an oscillating circuit, applying an electrically insulating layer on the electrically conductive means, applying second electrically conductive means on the electrically insulating layer which are to constitute another component of the oscillating circuit, and contacting the first electrically conductive means with the second electrically conductive means so that the oscillating circuit is formed. In this way, a structure providing the functionality required for the resonant frequency analysis is provided in only a few process steps.

Usefully, it is contemplated that the first electrically conductive means exhibit an exterior with windings and an interior with capacitive properties.

In addition, the production method is, in a particularly useful and simple manner, further developed in that the application of the first electrically conductive means and/or the electrically insulating layer and/or the second electrically conductive means is implemented using thin film technology.

Therefore, in the present invention, two different concepts for measuring a property around the implanted secondary device are used. In the first measurement principle, a change in the impedance or resonant frequency of an implanted oscillating circuit is analysed in a simple manner. This method does not require any active components in the organism so that problems with biocompatibility are reduced. In this case, the external field preferably operates in a range of one kHz to one GHz, more preferably in a range of 4 kHz to 120 kHz. The second measurement principle is based upon coupling electromagnetic power to the secondary device so that the actual detection of the state of the surroundings is assisted by active components. In this case, utilizing the above-named optimum frequency range for detecting impedance changes is less important; rather, the frequency can, for example, be selected so that as much energy as possible is transferred in the shortest possible period of time, or the used frequency range can be determined by entirely different criteria. First and foremost, it must be taken into consideration in this case that as per the technology according to Kraus and Lechner, electromagnetic alternating fields are deployed in connection with support metal osteosynthesis devices or joint endoprostheses. For this purpose, coil arrangements referred to as take-up or repeating coils are integrated into these implants, and their poles are electrically connected with implant sections acting as electrodes. One example of an osteosynthesis device using the described technology is disclosed in DE 10 2006 018 191 A1. The femoral head cap implants described in 10 2004 024 473 A1 are examples of the use of the technology in joint endoprosthetics. Therefore, if the coils outside of the organism are adjusted to the frequency range of 1 to 30 Hz, preferably 10 to 20 Hz, required for the technology according to Kraus and Lechner, on the one hand, this technology can be used, and, on the other hand, the power required for operating the measuring device can also be transferred into the system according to the invention.

Another potential field of application is the non-invasive monitoring of the bone growth in case of fractures treated with osteosynthesis material (e.g. osteosynthesis plate, marrow nail). Here, impedance changes which are also detected by means of electrodes deposited on an insulating intermediate layer may give information on the progress of the bone consolidation of the fracture. Thus, the repeated taking of X-rays can be avoided. The application of the insulating intermediate layer and the electrodes may also be carried out by means of thin film technology.

The field of drug targeting is another example in which the present invention can be usefully deployed owing to the double function of the magnetic field outside of the organism. Here, implants are provided with magnetically conductive properties in order for a concentration of the magnetic field in the area of the implantable device to result from external magnetic fields. If the implantable device is positioned in the region of flowing body fluids, i.e., in a blood vessel, a concentration of paramagnetic nanoparticles comprising active agents or cells coupled thereto can be brought about in the region of the implantable device by administering said nanoparticles. Both the first embodiment of the present invention comprised of a mere electric oscillating circuit, as well as the second embodiment comprised of active components can be combined with drug targeting technology. On the one hand, the electromagnetic field generated outside the organism can be concentrated in the area of the implant for the purpose of concentrating agents or cells, and on the other hand, either the resonant frequency will be monitored, or it will be used provide the power for active components in the area of the implant.

The invention will now be described by way of example with the aid of preferred embodiments with reference to the accompanying drawings in which:

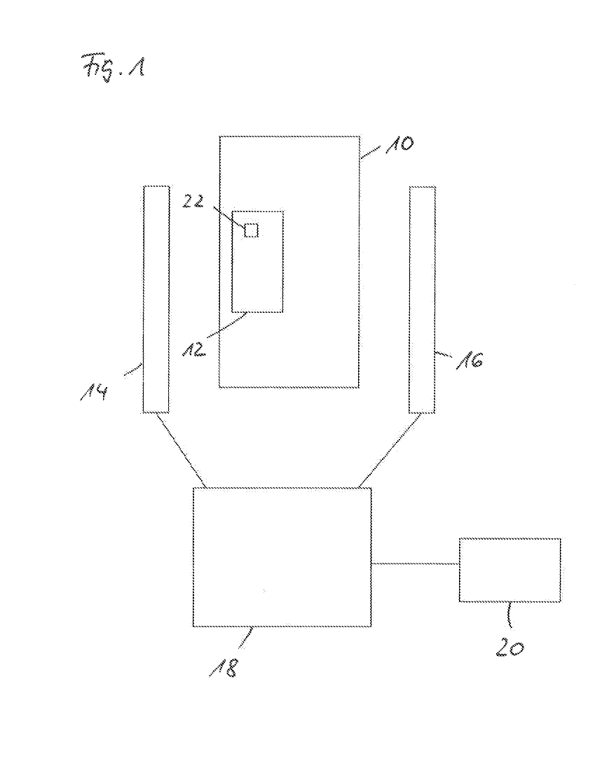

FIG. 1 shows a first embodiment of a system according to the invention;

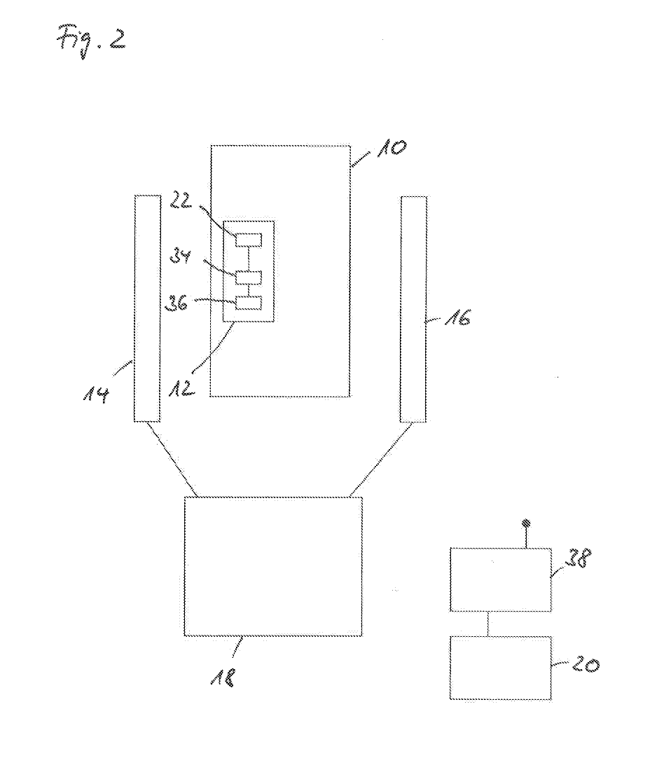

FIG. 2 shows a second embodiment of a system according to the invention, and

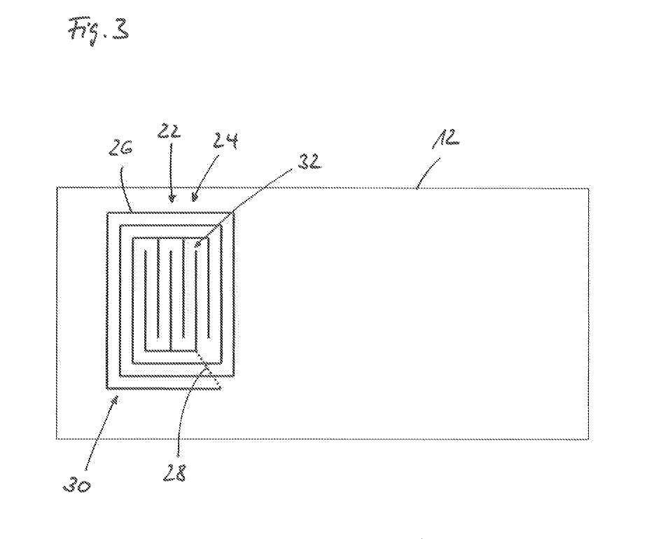

FIG. 3 shows a secondary device to be used in a system according to the invention.

In the following description of the drawings the same numerals designate the same or comparable components.

FIG. 1 shows a first embodiment of a system according to the invention. A secondary device 12 is implanted in an organism 10, in particular, a living human body. A coil arrangement 14, 16 is provided outside of the organism which acts as a primary device and is capable of generating an electromagnetic field in the vicinity of the secondary device 12. The coil arrangement may, for example, be realised by Helmholtz coils 14, 16 as shown, or in any other way. It is important that an electromagnetic field exists in the vicinity of the secondary device 12. The coils 14, 16 outside of the organism are supplied with power by a function current generator 18.

The secondary device 12 is now provided with electrically conductive means 22 constituting an electric oscillating circuit. The impedance and resonant frequency of this electric oscillating circuit depend on the state of its surroundings, particularly on the tissue condition, the presence or absence of biofilms, or any other parameters reflecting the conditions in the organism 10. Now if the frequency of the function current generator 18 is, for example, adjusted so that it corresponds to the resonant frequency of the oscillating circuit constituted by the electrically conductive means 22, this resonance state can be monitored by the evaluation device 20. If the resonant frequency of the oscillating circuit inside the organism shifts, i.e. if there are changes in the surroundings of the secondary device 12, this will be detected by the evaluation device 20. This may suggest an excessive cell growth if the secondary device 12 is, for example, a stent. The formation of biofilms on implants forming the secondary device 12 can also be detected at an early stage. It is not necessarily required to operate the external alternating field at the resonant frequency of the oscillating circuit; other spectral components are also influenced by the changing conditions in the vicinity of the secondary device.

FIG. 2 shows a second embodiment of a system according to the invention. In contrast to the embodiment according to FIG. 1, the detection of the state by the secondary device 12 is not necessarily based on monitoring a resonance state. Rather, the secondary device 12 is provided with a measuring device 34 and a transmitter device 36. The measuring device 34 and the transmitter device 36 are supplied with power via the electrically conductive means 22. The electrically conductive means 22 draws said power from the electromagnetic field generated by the coil arrangement 14, 16 outside of the organism. The measuring device 34 may comprise any kind of sensor technology to detect status parameters in the vicinity of the secondary device 12. For example, impedances may again be detected, or other parameters, such as the pH-value, may also be detected. The measuring device 34 usefully comprises an ion-sensitive field-effect transistor in the latter case. The signals transmitted by the transmitter device 36 are received by a receiver device 38 which relays them to an evaluation device 20.

The devices described above in connection with the embodiments according to FIGS. 1 and 2, particularly the electric means 22, the measuring device 34, the transmitter device 36, the receiver device 38, the evaluation device 20, and the function current generator 18, may be realised individually as well as in an integrated form. For example, it is possible in the embodiment according to FIG. 2 that the electric means 22, the measuring device 34 and the transmitter device 36 are realised in a partially or fully integrated manner. The receiver device 38 and/or the evaluation device 20 comprising the function current generator 18 may also be fully or partially integrated.

FIG. 3 shows a secondary device to be used in a system according to the invention. The secondary device 12 carries electrically conductive means 22 forming an electric oscillating circuit 24. The exterior 30 of the electrically conductive means 22 has windings, i.e. inductive properties, while the interior 32 has capacitive properties. In order to realise the oscillating circuit, for example, the conductor structure realised by continuous lines is first deposited on the electrically insulating secondary device 12. This conductor structure is referred to as first electrically conductive means 26. If the secondary device 12 is not already insulated, an insulating layer is applied to the secondary device 12 before the first electrically conductive means 26 is applied. After the application of the first electrically conductive means 26, an insulating layer is deposited on the first electrically conductive means 26. Second electrically conductive means 28 are then applied on the insulating layer not visible here. In order to contact the electrically conductive means 26, 28 for an electric oscillating circuit 24 to be formed, two contacts are established between the first electrically conductive means 26 and the second electrically conductive means 28, namely one at one pole of the capacitors connected in parallel in the interior 32 of the arrangement and another at the exterior pole of the inductive exterior 30. Various thin film technologies can be used to realise the layer structure described, which may also be employed in combination, for example, physical vapour deposition (PVD) and chemical vapour deposition (CVD). Sputtering techniques may also be used.

The features of the invention disclosed in the above description, in the drawings as well as in the claims may be important for the realisation of the invention individually as well as in any combination.

### LIST OF NUMERALS

10 organism
12 secondary device
14 coil arrangement
16 coil arrangement
18 function current generator
20 evaluation device
22 electrically conductive means
24 electric oscillating circuit
26 electrically conductive means
28 electrically conductive means
30 exterior
32 interior
34 measuring device
36 transmitter device
38 receiver device

1. A system for detecting measured values in or on an organism, comprising:
at least one secondary device attachable to the organism or implantable in the organism, the impedance of which depends on a state of the surroundings of the implantable secondary device,
a coil arrangement placeable outside the organism as a primary device for generating an electromagnetic alternating field in the vicinity of the secondary device in an implanted state, and
an evaluation device placeable outside of the organism for detecting and analysing measured values depending on the impedance of the secondary device,
wherein the secondary device comprises an electrically conductive element applied using thin film technology.

2. The system according to claim 1, wherein the secondary device comprises an electric oscillating circuit the impedance and resonant frequency of which depend on the state of the surroundings of the secondary device.

3. The system according to claim 1, wherein
the electrically conductive element comprises a first electrically conductive means (26) portion applied to the implantable secondary device which constitutes a component of an oscillating circuit,
an electrically insulating layer is applied on the first electrically conductive portion,
second electrically conductive portion which constitutes another component of the oscillating circuit applied on the electrically insulating layer, and
the first electrically conductive portion is in contact with the second electrically conductive portion so that the oscillating circuit is formed.

4. The system according to claim 3, wherein the first electrically conductive portion comprises an exterior with windings and an interior with capacitive properties.

5. The system according to claim 4, wherein
the secondary device includes a measuring device detecting measured values depending on the state of tissue surrounding the secondary device,
the secondary device includes a transmitter device transmitting signals depending on the measured values, and
a receiver device placeable outside the organism receives signals transmitted by the transmitter device and supplies the signals to the evaluation device.

6. The system according to claim 5, wherein the transmitter device comprises at least one RFID transponder.

7. The system according to claim 6, wherein a readable information content of the RFID transponder is changeable depending on measured values provided by the measuring device.

8. The system according to claim 5, wherein a plurality of RFID transponders is provided which are activatable or deactivatable depending on measured values provided by the measuring device.

9. A method for producing a secondary device in which an electric voltage is inducible by a primary device, comprising the steps of:
providing an implant;
applying first electrically conductive portion of an oscillating circuit on the implant;
applying an electrically insulating layer on the electrically conductive means;
applying second electrically conductive portion of the oscillating circuit on the electrically insulating; and
contacting the first electrically conductive portion with the second electrically conductive portion so that the oscillating circuit is formed.

10. The method according to claim 9, wherein the first electrically conductive portion includes an exterior with windings and an interior with capacitive properties.

11. The method according to claim 10, wherein the application of at least one of the first electrically conductive portion, the electrically insulating layer, and the second electrically conductive portion is implemented using thin film technology.
